(19) United States
(12) Patent Application Publication
(10) Pub. No.: US 2004/0077940 A1
(43) Pub. Date: Apr. 22, 2004

(54) Instrument guide for use with a tracking system

(76) Inventors: Thomas C. Kienzle III, Lake Forest, IL (US); Jon T. Lea, Hampstead, NH (US); Thomas H. Peterson, Wilmington, MA (US)

Correspondence Address: MCANDREWS HELD & MALLOY, LTD, 500 WEST MADISON STREET, SUITE 3400, CHICAGO, IL, 60661

(21) Appl. No.: 10/677,538

(22) Filed: Oct. 2, 2003

Related U.S. Application Data
(60) Provisional application No. 60/417,865, filed on Oct. 11, 2002.

(51) Int. Cl. A61B 5/05

(52) U.S. Cl. 600/424

(57) ABSTRACT

An instrument guide for use with an elongated instrument and a computer assisted surgery tracking system. The instrument guide includes a handle assembly, a localizer assembly connected to the handle assembly at a central bore, and at least one guide sleeve. The guide sleeve has a tip end, an attachment end, and a sleeve bore. The attachment end is configured to be front loaded into the central bore and rigidly and removably connected to the central bore. The front loaded at least one guide sleeve is inserted into the central bore such that the tip end does not pass through the central bore. The guide sleeve receives the elongated instrument at the attachment end, and the sleeve bore has a fixed and known position relative to the localizer assembly such that the localizer assembly is used to determine the trajectory of the sleeve bore.

This application is related to, and claims priority from, Provisional Application No. 60/417,865 filed Oct. 11, 2002, titled “Drill Guide With Interchangeable Sleeves For Use With A Computer Assisted Surgery System,” the complete disclosure of which is incorporated herein by reference in its entirety.

### BACKGROUND OF THE INVENTION

The present invention relates to an instrument guide for use with a localizer tracking system. More particularly, certain embodiments of the present invention relate to an adjustable drill guide sleeve that is front loaded into a surgical drill guide such that it is located at a known and fixed position relative to a localizing device.

During surgical operations, it is beneficial to be able to track the trajectory of a surgical instrument, such as a drill bit, into a surgical site on a patient's body in order to ensure that the instrument is directed into the appropriate point in the body. In order to better track the orientation and trajectory of a drill bit entering a surgical site, surgical drills are often used with tracked drill guides. Tracked drill guides typically include a localizing device and a handle that are connected at a cylindrical collar. The collar has a central bore that receives a guide sleeve. The guide sleeve is rear loaded into the central bore by passing the entire guide sleeve through the central bore from a back end to a front end. The localizing device is part of a localizing assembly and can communicate with other localizing devices. The localizing assembly may utilize optical or electromagnetic localizers, for example. The localizing assembly communicates with a computer that displays an image of the surgical site.

The guide sleeve includes a bore that extends from the front end to the back end of the drill guide and that has a fixed and known position relative to the localizing device. The localizing device communicates with other localizing devices in the localizing assembly to calculate the pose of the drill guide relative to the surgical site. The localizing assembly communicates the pose of the drill guide to the computer and the computer calculates the pose of the drill guide bore. The computer then recreates the position of the drill guide on the image such that a surgeon can know the position and orientation of the guide sleeve bore to the surgical site and thus the trajectory of the drill bit.

In operation, the surgeon views the a graphic representation of the guide sleeve bore superimposed on an image of the surgical site in order to determine the appropriate position and orientation of the guide sleeve for surgery on the imaged surgical site. Once the guide sleeve bore is shown on the image to be in the appropriate position, the drill bit is inserted into the guide sleeve through a rear end of the drill guide and into the surgical site. The surgeon is able to track the orientation and trajectory of the drill bit by viewing the position of the drill guide bore on the computer image during the course of surgery.

In some cases, the drill guide incorporates a means to change the diameter of the bore of the drill guide to accommodate drill bits of different sizes. For example, a large inner diameter guide sleeve may be incorporated into the drill guide. The large diameter guide sleeve has an entry end near the handle of the drill guide, and a tip end that is directed toward the surgical site. The large diameter guide sleeve is large enough to accommodate insertable guide sleeves of varying smaller inner diameters. The smaller diameter guide sleeves are inserted into the large diameter guide sleeve from its entry end.

The conventional drill guide suffers from a number of drawbacks. First, inserting smaller diameter guide sleeves into the larger diameter guide sleeves can be a cumbersome method to accommodate instruments of different sizes. Also, it requires that the length and diameter of the guide sleeves be large enough to accommodate the largest object intended to be passed though it. Therefore, even the smallest diameter drill bits (or guide pin, K-wire, screw, etc.) will be required to pass through a relatively thick guide sleeve that is cumbersome and does not facilitate the use of the smallest possible incision. Further, all drill bits are required to be longer than the guide sleeve, which can be problematic for narrow drill bits. Also, this requires that for even the most delicate or well exposed body parts, the surgeon must use a long, wide, and unnecessarily cumbersome drill guide to drill a small hole.

Additionally, a surgical procedure often requires that a drill bit be directed into the surgical site at a particular angle. Therefore, it is desirable to be able to attach a bore foot to the tip end of the guide sleeve bore such that the bore foot is positioned relative to the guide sleeve bore at the particular angle. In operation, the bore foot engages the surgical site such that the guide sleeve bore is oriented to the surgical site at the particular angle, and thus the drill bit is inserted into the surgical site at the particular angle. However, a guide sleeve cannot be rear loaded through the central bore with a bore foot already attached because the bore foot is too large to fit through the collar. Therefore, the guide sleeve bore of a conventional drill guide cannot conveniently be used with an angled bore foot.

Therefore, a need exists for an improved drill guide that can accommodate a guide sleeve bore that includes attachments.

### BRIEF SUMMARY OF THE INVENTION

Certain embodiments of the present invention include an instrument guide for use with an elongated instrument and a computer assisted surgery tracking system. The instrument guide includes a handle assembly, a localizer assembly connected to the handle assembly at a central bore, and at least one guide sleeve. The guide sleeve has a tip end, an attachment end, and a sleeve bore. The attachment end is configured to be front loaded into the central bore and rigidly and removably connected to the central bore. The front loaded at least one guide sleeve is inserted into the central bore such that the tip end does not pass through the central bore. The guide sleeve receives the elongated instrument at the attachment end, and the sleeve bore has a fixed and known position relative to the localizer assembly such that the localizer assembly is used to determine the trajectory of the sleeve bore.

Certain embodiments of the present invention include a computer assisted surgical tracking system. The tracking system includes a drill bit, a computer that displays an image of a surgical site, and a drill guide. The drill guide has a handle assembly, a localizer assembly connected to the handle assembly at a central bore, and at least one guide sleeve. The guide sleeve has a tip end, an attachment end, and a sleeve bore. The attachment end is configured to be front loaded into the central bore and rigidly and removably connected to the central bore. The front loaded at least one guide sleeve is inserted into the central bore such that the tip end does not pass through the central bore. The guide sleeve receives the drill bit at the attachment end and the sleeve bore has a fixed and known position relative to the localizer assembly such that the localizer assembly communicates the position of the guide sleeve to the computer. The computer displays the position and trajectory of the guide sleeve on the image relative to the surgical site.

Certain embodiments of the present invention include a method for tracking a surgical procedure. The method includes taking an image of a surgical site and storing the image on a computer system, providing a surgical instrument, and providing an instrument guide having a localizer assembly and a handle assembly connected at a central bore and a guide sleeve having a tip end, an attachment end, and a sleeve bore. The guide sleeve has a fixed and known position relative to the localizer assembly. The method further includes front loading the attachment end of the guide sleeve such that the tip end of the guide sleeve does not pass through the central bore, calculating the position of the guide sleeve and displaying the position of the guide sleeve on the image relative to the surgical site by communication between the localizer assembly and the computer system, and inserting the surgical instrument into the guide sleeve at the attachment end such that the surgical instrument emerges from the tip end to engage the surgical site.

### BRIEF DESCRIPTION OF SEVERAL VIEWS OF THE DRAWINGS

FIG. 1 is an isometric view of a drill guide formed according to an embodiment of the present invention.

FIG. 2 is an exploded isometric view of a drill guide of FIG. 1.

FIG. 3 is an isometric view of a drill guide with a bore foot formed according to an embodiment of the present invention.

FIG. 4 is an isometric view of a computer assisted surgery tracking system formed according to an embodiment of the present invention.

The foregoing summary, as well as the following detailed description of certain embodiments of the present invention, will be better understood when read in conjunction with the appended drawings. For the purpose of illustrating the invention, there is shown in the drawings, certain embodiments. It should be understood, however, that the present invention is not limited to the arrangements and instrumentality shown in the attached drawings.

### DETAILED DESCRIPTION OF THE INVENTION

The present invention is preferably operated in conjunction with an image guided surgery system such as is disclosed in U.S. Pat. No. 6,748,802 entitled “Computer Assisted Targeting Device for Use in Orthopedic Surgery” or U.S. Pat. No. 5,829,444 entitled “Position Tracking and Imaging System for Use in Medical Applications,” the disclosures of which we hereby incorporated by reference.

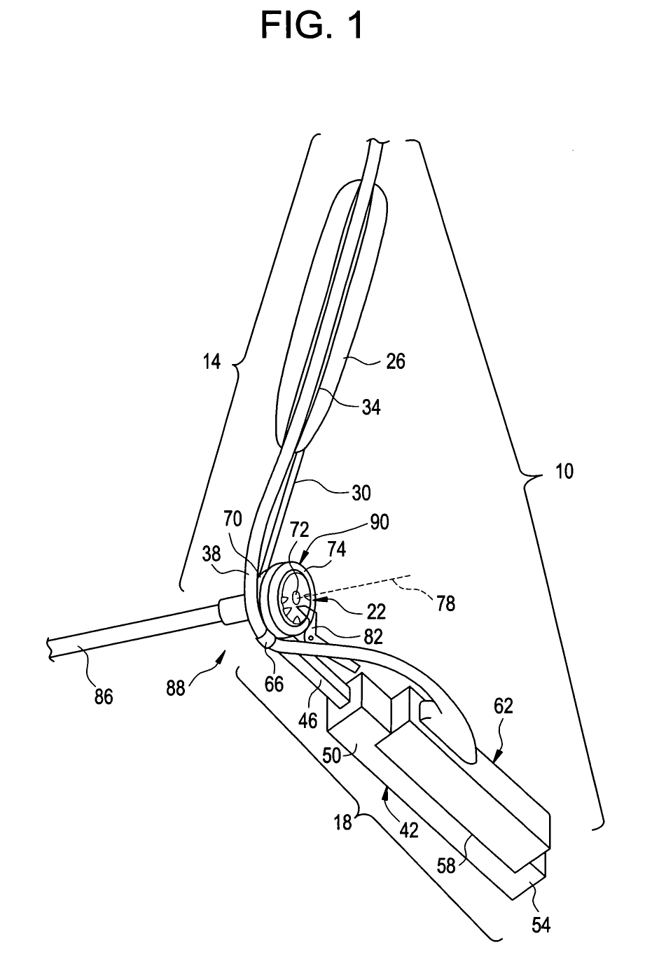

FIG. 1 is an isometric view of an instrument guide or drill guide 10 formed according to an embodiment of the present invention. The drill guide 10 includes a handle assembly 14 connected to a localizer assembly 18 at a collar section 22. The handle assembly 14 includes a handle 26 mounted to a handle stem 30. The handle stem 30 extends from the collar section 22. The localizer assembly 18 includes a mounting block 42 mounted to a localizer stem 46. The localizer stem 46 extends from the collar section 22. The mounting block 42 includes a block 50 and a foot 54 separated by a gap 58. The mounting block 42 receives a localizer or an electromagnetic receiver 62 in the gap 58 such that the receiver 62 is secured between the block 50 and the foot 54.

The collar section 22 includes a circular first collar 70 connected to a circular second collar 74. The first and second collars 70 and 74 are concentrically aligned with each other in order to define a central bore 72 along a longitudinal axis 78. The handle stem 30 is connected to the first collar 70 and the localizer stem 46 is connected to the second collar 74. The first and second collars 70 and 74 rotate relative to each other about the longitudinal axis 78. The collar section 22 includes a locking mechanism 82 that may be manipulated to secure the first and second collars 70 and 74 to each other such that the first and second collars 70 and 74 may not be rotated relative to each other. The collar section 22 receives a guide sleeve 86 that extends from a front end 88 of the drill guide 10. The guide sleeve 86 is connected to the collar section 22 in order to receive an instrument, such as a drill bit or guide pin, through the central bore 72 from a rear end 90 of the drill guide 10.

The receiver 62 is configured to receive electromagnetic signals as part of an electromagnetic localizer system. Alternatively, the receiver 62 may be an optical localizer such as a light emitting diode that communicates with an optical tracking system. In an electromagnetic localizer system, a transmitter (not shown) that is located in a fixed position relative to a surgical site generates an electromagnetic field to communicate with the receiver 62. The receiver 62 and the transmitter are electrically connected to a computer system (not shown) such that the computer may calculate or determine the position of the receiver 62 relative to the transmitter (and thus the surgical site) based on the electromagnetic communications therebetween. The receiver 62 is connected to the computer system by a cord 38, as is the transmitter (now shown). Alternatively, a wireless system may be used. The cord 38 extends from the receiver 62 to the collar section 22 and is secured to the collar section 22 by a clip 66. The handle 26 has a groove 34 that receives the cord 38 extending along the stem 30 from the localizer assembly 18. The groove 34 may be machined on the surface of the handle 26 or may be routed through the interior of the handle 26. The cord 38 is secured in the clip 66 and the handle 26 such that the cord 38 does not hang loosely from the drill guide 10. Thus, a surgeon can manipulate the drill guide 10 with the cord 38 out of the way of the operation.

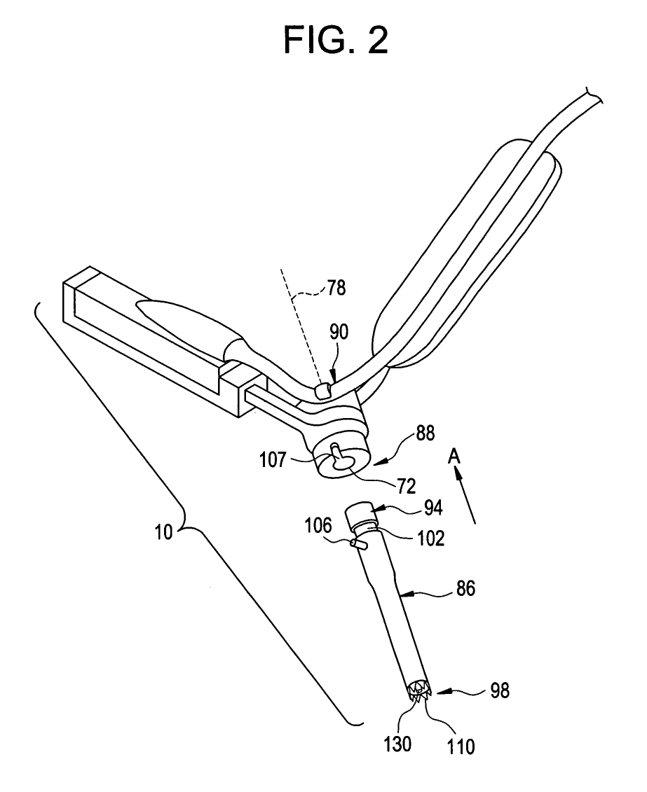

FIG. 2 is an exploded isometric view of a drill guide 10 of FIG. 1. The guide sleeve 86 is detached from the drill guide 10. In order to prepare the drill guide 10 for use during an operation, the drill guide 10 is first calibrated with a calibration shaft (not shown). With the guide sleeve 86 removed from the drill guide 10, the calibration shaft is inserted into the central bore 72 of the drill guide 10. The positions of the ends of the calibration shaft are located relative to the localizer system by touching them to a reference point of known location. The pose of the trajectory of the drill guide 10 relative to the localizer assembly is then stored in the computer system. Alternatively, any calibration method may be used to determine the pose of the trajectory of the drill guide 10.

The guide sleeve 86 is generally cylindrical in shape and includes an attachment end 94 and a tip end 98 extending from opposite sides of a bore 130. The bore 130 has an inner diameter of a size appropriate to accommodate a drill bit, guide pin, or other insertable instrument. The tip end 98 of the guide sleeve 86 is preferably in the shape of a tube and has serrations 110. The serrations 110 are configured to engage bone such that when the guide sleeve 86 is pushed against a surgical site during surgery, the guide sleeve 86 does not slip.

The attachment end 94 of the guide sleeve 86 includes a circumferential groove 102 and a radially oriented pin 106. In operation, the attachment end 94 of the guide sleeve 86 is inserted in the direction of arrow A into the central bore 72 through the front end 88 of the collar section 22. That is to say, the guide sleeve 86 is front loaded into the central bore 72 such that the tip end 98 does not pass through the central bore 72. The central bore 72 includes a retaining ring (not shown) that is snapably received into the circumferential groove 102 of the guide sleeve 86 to prevent the guide sleeve 86 from sliding within the central bore 72 along the longitudinal axis 78. Additionally, the pin 106 is received within a slot 107 in the central bore 72 when the guide sleeve 86 is inserted into the central bore 72. The pin 106 engages the slot 107 in the central bore 72 to prevent rotation of the guide sleeve 86 about the longitudinal axis 78 relative to the central bore 72. Alternatively, the side of the guide sleeve 86 may have a flat surface, or other keying feature, that mates with a flat surface, or other keying feature, on the central bore 72, such that rotation of the guide sleeve 86 around its longitudinal axis 78 relative to the collar section 22 is prevented. Alternatively, the guide sleeve 86 may have several grooves along the attachment end 94 such that the guide sleeve 86 may be threadably inserted into the central bore 72. Alternatively, any number of other mechanisms may be employed to rigidly attach the guide sleeve 86 in the central bore 72.

Additionally, guide sleeves 86 of different lengths and inner diameters may be interchangeably inserted into the central bore 72 as long as the guide sleeves 86 have an attachment end 94 that is insertable into the central bore 72. Thus, a surgeon may easily interchange, and rigidly secure, different guide sleeves 86 into the central bore 72 to accommodate instruments of varying size. Furthermore, guide sleeves 86 of different sizes and shapes may be interchangeably inserted into the central bore 72 as long as the guide sleeves 86 have an attachment end 94 that is insertable into the central bore 72. For example, guide sleeves may have different lengths or have different non-cylindrical or partially cylindrical shapes. In this respect, the drill guide may include a smart coupler for indicating the specific guide sleeve that is connected to the instrument guide. Examples of such smart couplers can be found in U.S. Pat. Nos. 5,693,042 and 6,402,743, the disclosures of which we hereby incorporated by reference.

In operation, the serrations 110 of the tip end 98 are positioned against a surgical site such that the guide sleeve 86 maintains its position on the bone. The drill bit or guide pin is then inserted into the rear end 90 of the central bore 72, through the bore 130 of the guide sleeve 86, and out the tip end 98 of the guide sleeve 86 to enter the bone. Thus, the guide sleeve 86 serves to maintain the orientation and trajectory of the drill bit or guide pin to the bone.

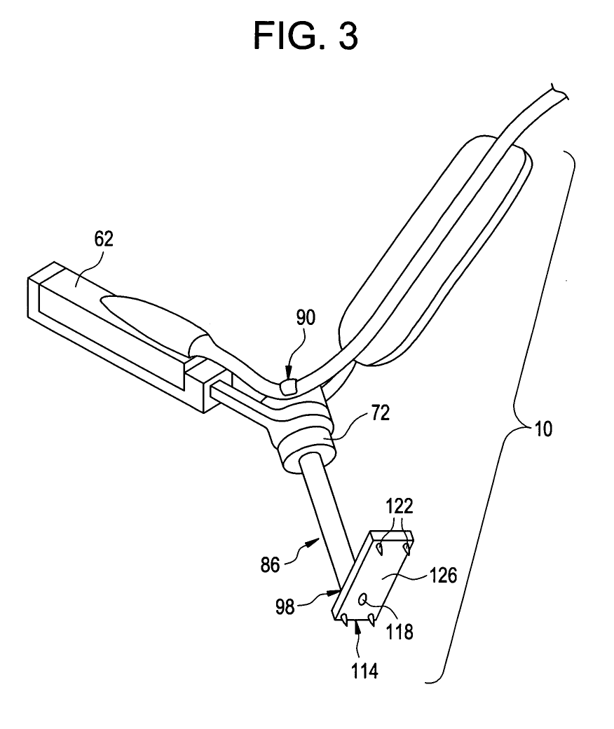

FIG. 3 is an isometric view of the drill guide 10 with a bore foot 114 formed according to an embodiment of the present invention. The bore foot 114 is a thin, rectangular plate connected to the tip end 98 of the guide sleeve 86. The bore foot 114 is in a fixed and known position relative to the receiver 62 such that the computer system may calculate or determine the position of the bore foot 114 and display a corresponding representation of the bore foot 114 relative to the surgical site. The bore foot 114 has a hole 118 that is aligned with the bore 130 of the guide sleeve 86 such that an instrument may be extended out the tip end 98 through the hole 118. The bore foot 114 also includes pointed tacks 122 that extend outward from a bottom side 126 thereof. The bore foot 114 is oriented at a known angle relative to the bore 130 (FIG. 2) of the guide sleeve 86.

In operation, when the bottom side 126 of the bore foot 114 is placed flush against the bone at the surgical site, the tacks 122 engage the bone such that the bore foot 114 retains its position on the bone. The drill bit is then inserted into the rear end 90 of the central bore 72 such that it emerges from the bore 130 (FIG. 2) of the guide sleeve 86 and the hole 118 of the bore foot 114 to enter the bone at the desired angle. Typically, the angle is 95 or 135 degrees depending on the procedure being performed, but the bore foot 114 may be connected to the bore 130 at any angle that is required.

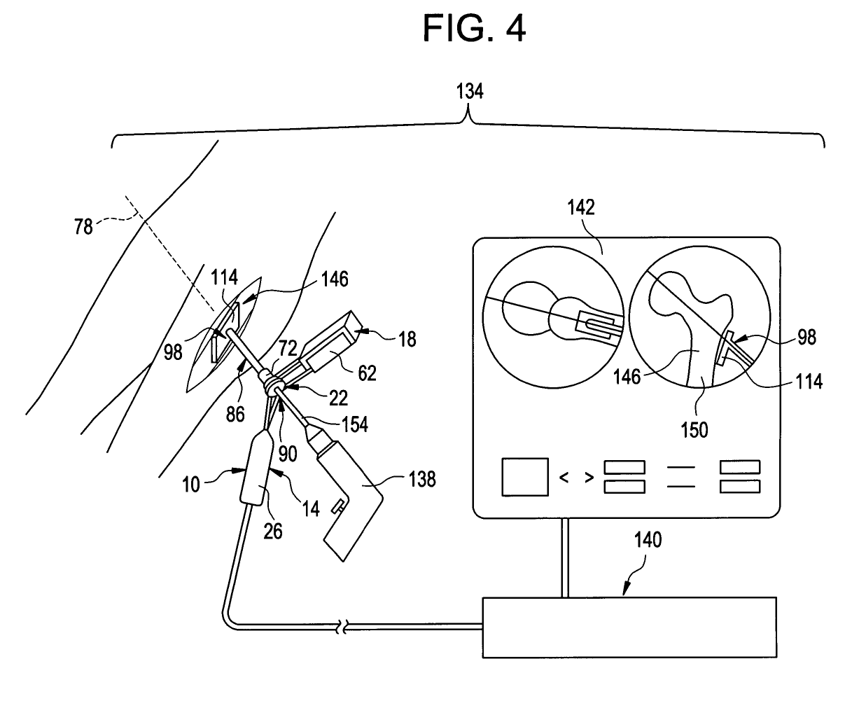

FIG. 4 is an isometric view of a computer assisted surgery tracking system 134 formed according to an embodiment of the present invention. The system includes the drill guide 10, a drill 138 with a drill bit 154, a computer 140 with a display 142, and a surgical site revealing a patient's bone 146. First, at least one image 150 is taken of the bone 146 upon which the surgeon is going to operate. The image 150 may be an X-ray, a CT Scan, an MRI or any other appropriate image. The image 150 is stored on the computer 140 and shown on the display 142. The drill guide 10 is then calibrated as described above such that the pose of the trajectory of the drill guide 10 relative to the localizer system is stored in the computer 140.

Once the drill guide 10 is properly calibrated and tracked, the surgeon attaches a desired guide sleeve 86 to the drill guide 10. The position of the guide sleeve 86 is fixed and known relative to the receiver 62. The receiver 62 on the drill guide 10 communicates with the localizer system and the computer 140 such that the computer 140 can calculate or determine, and show on the display 142, the position of the drill guide 10 relative to the bone 146. Thus, the surgeon can track the movement of the drill guide 10 relative to the image of the bone 146 on the display 142 before and during surgery. Because the position of the guide sleeve 86 is fixed and known relative to the receiver 62, the computer 140 can calculate or determine and display the position of the tip end 98 and the bore foot 114 relative to the bone 146. While the guide sleeve 86 is shown with the bore foot 114 for the purposes of FIG. 4, the computer assisted surgery system 134 may be used to track a guide sleeve 86 having only a serrated tip end 98 (FIG. 2).

The surgeon then positions the receiver 62 in the proper location in the field of surgery. The surgeon disengages the locking mechanism 82 (FIG. 1) on the collar section 22 such that the localizer assembly 18 may be rotated about the longitudinal axis 78 to a desired position relative to the handle 26 and the field of surgery. The ability to freely rotate the receiver 62 to a desired position is especially beneficial where the localizer system is an electromagnetic system. For example, the surgeon can move the receiver 62 to a position where metal components or other electromagnetic devices cannot interfere with the receiver's 62 ability to receive signals. Likewise, the surgeon may move the handle assembly 14 to a desirable position.

Once the drill guide 10 is properly configured and calibrated, the surgeon may begin performing surgery. The drill guide 10 is positioned at the appropriate location along the bone 146. The position of drill guide 10 and its guide sleeve 86 relative to the bone 146 is shown on the image 150 of the bone 146 on the computer display 142 in order for the surgeon to verify that the bore 130 (FIG. 2) of the guide sleeve 86 is appropriately oriented along the bone 146. The display of the bore foot 114 on the image 150 also allows the surgeon to verify the angle at which the drill bit 154 will enter the bone 146. Once the surgeon is satisfied that the drill guide 10 is in the proper position along the bone 146, the surgeon inserts the drill bit 154 of the drill 138 into the rear end 90 of the central bore 72. The surgeon extends the drill bit 154 on through the bore 130 of the guide sleeve 86 and out the tip end 98 and the bore foot 114 until the drill bit 154 engages the bone 146. The surgeon is able to track the position of the drill guide 10 on the display 142 throughout the surgery in order to be sure that the drill bit 154 is always engaging the bone 146 at the desired orientation and trajectory.

In an alternative embodiment, the guide sleeve 86 may include a guide jig or some other attachment or feature besides a bore foot 114 along the bore 130. The position of the attachment is fixed and known relative to the receiver 62. Alternatively, the instrument inserted into the drill guide may be a guide pin, screwdriver, saw blade, or any other elongated instrument besides a drill bit. Additionally, the guide may be an instrument guide that may be used with different instruments besides a surgical drill. Alternatively, instead of a guide sleeve 86, a cutting block, templating device, or other instrument may be attached to the drill guide 10 and its position is fixed and know relative to the receiver 62. Furthermore, the guide may be used with non-medical instruments.

The drill guide of the different embodiments provides several advantages. One or more drill sleeves may be interchangeably attached to the central bore. Each drill sleeve is rigidly and removably attached to the guide sleeve such that the bore of each drill sleeve defines the same trajectory as tracked by the localizer system. Therefore, the surgeon can interchange guide sleeves of varying inner diameters (to accommodate instruments of varying diameters) without affecting the tracking of the guide sleeves. Further, the surgeon does not have to insert smaller diameter guide sleeves within a larger diameter guide sleeve to alter the size of a guide sleeve bore. Additionally, the surgeon can interchange guide sleeves of varying length to accommodate the varying depths of soft tissue encountered around the site of surgery.

Additionally, each guide sleeve is connected to the central bore by front loading the attachment end of the guide sleeve into the central bore. That is to say, the guide sleeves do not entirely have to be passed through the central bore, i.e., be rear loaded, in order to be connected to the central bore. Therefore, a guide sleeve having an attachment that is larger than the diameter of the central bore may still be connected to the central bore. For example, a guide sleeve having a bore foot could not be rear loaded through the central bore because the bore foot would not fit through the central bore. However, with the present invention, the guide sleeve is front loaded into the central bore such that the bore foot need not pass through the central bore in order to connect the guide sleeve to the central bore. Thus, the drill guide of the present invention may easily be used with guide sleeves having attachments.

While the invention has been described with reference to certain embodiments, it will be understood by those skilled in the art that various changes may be made and equivalents may be substituted without departing from the scope of the invention. In addition, many modifications may be made to adapt a particular situation or material to the teachings of the invention without departing from its scope. Therefore, it is intended that the invention not be limited to the particular embodiment disclosed, but that the invention will include all embodiments falling within the scope of the appended claims.

1. An instrument guide for use with an elongated instrument and a computer assisted surgery tracking system, comprising:
a localizer assembly having a central bore;
a handle assembly connected to said localizer assembly; and
at least one guide sleeve, said at least one guide sleeve having a tip end, an attachment end, and a sleeve bore, said attachment end being configured to be front loaded into said central bore and rigidly and removably connected to said central bore, said front loaded at least one guide sleeve is inserted into said central bore such that said tip end does not pass through said central bore, said at least one guide sleeve receiving said elongated instrument at said attachment end and said sleeve bore having a fixed and known position relative to said localizer assembly such that said localizer assembly is used to determine the trajectory of said sleeve bore.

2. The instrument guide of claim 1, wherein said front loaded at least one guide sleeve is inserted into said central bore such that said attachment end extends into said central bore.

3. The instrument guide of claim 1, wherein said at least one guide sleeve includes a foot that is oriented to said guide sleeve at a desired angle, said foot engaging a surgical site such that said at least one guide sleeve is oriented to said surgical site at said desired angle.

4. The instrument guide of claim 1, wherein said tip end has serrations for engaging a surgical site and maintaining the position of said at least one guide sleeve at said surgical site.

5. The instrument guide of claim 1, wherein said localizer assembly includes a receiver that has a fixed and known position relative to said at least one guide sleeve, said receiver communicating with a computer such that said computer is used to determine the position of said at least one guide sleeve and displays the position of said at least one guide sleeve relative to a desired surgical site.

6. The instrument guide of claim 1, wherein said attachment end of said at least one guide sleeve includes a groove and a pin, wherein when said at least one guide sleeve is inserted into said central bore, said groove receiving a ring in said central bore and said pin being received in a hole in said central bore to rigidly maintain said at least one guide sleeve within said central bore.

7. The instrument guide of claim 1, wherein said elongated instrument is a drill bit.

8. The instrument guide of claim 1, wherein said at least one guide sleeve comprises a plurality of guide sleeves having varying inner diameters.

9. The instrument guide of claim 1, wherein said localizer assembly includes an electromagnetic receiver that communicates with an electromagnetic transmitter to determine the position of said receiver relative to a surgical site, said receiver and transmitter communicating with a computer such that said computer determines the position of said instrument guide relative to said surgical site.

10. The instrument guide of claim 1, wherein said handle assembly and said localizer assembly are movable relative to each other such that said handle and localizer assemblies may be moved to desirable positions.

11. The instrument guide of claim 1, wherein said at least one guide sleeve comprises a plurality of guide sleeves of varying lengths.

12. The instrument guide of claim 1, wherein said at least one guide sleeve comprises a feature, and there is a fixed and known relationship between said feature and said localizer assembly, such that said localizer assembly is used to determine the position of said feature.

13. A computer assisted surgical tracking system, comprising:
a drill bit;
a computer that displays an image of a surgical site; and
a drill guide, said drill guide having a handle assembly, a localizer assembly connected to said handle assembly at a central bore, and at least one guide sleeve, said at least one guide sleeve having a tip end, an attachment end, and a sleeve bore, said attachment end being configured to be front loaded into said central bore and rigidly and removably connected to said central bore, said front loaded at least one guide sleeve is inserted into said central bore such that said tip end does not pass through said central bore, said at least one guide sleeve receiving said drill bit at said attachment end and said sleeve bore having a fixed and known position relative to said localizer assembly such that said localizer assembly communicates the position of said at least one guide sleeve to said computer, said computer displaying the position and trajectory of said guide sleeve on said image relative to said surgical site.

14. The tracking system of claim 13, wherein said front loaded at least one guide sleeve is inserted into said central bore such that said attachment end extends into said central bore.

15. The tracking system of claim 13, wherein said at least one guide sleeve includes an attachment, said attachment having a fixed and known position relative to said localizer assembly.

16. The tracking system of claim 15, wherein said attachment is a foot that is oriented to said guide sleeve at a desired angle, said foot engaging said surgical site such that said at least one guide sleeve is oriented to said surgical site at said desired angle.

17. The tracking system of claim 13, wherein said tip end has serrations for engaging said surgical site and maintaining the position of said at least one guide sleeve at said surgical site.

18. The tracking system of claim 13, wherein said attachment end of said at least one guide sleeve includes a groove and a pin, wherein when said at least one guide sleeve is inserted into said central bore, said groove receiving a ring in said central bore and said pin being received in a hole in said central bore to rigidly maintain said at least one guide sleeve within said central bore.

19. The tracking system of claim 13, wherein said at least one guide sleeve comprises a plurality of guide sleeves having varying diameters.

20. The tracking system of claim 13, wherein said localizer assembly includes an electromagnetic receiver that communicates with an electromagnetic transmitter to determine the position of said receiver relative to said surgical site, said receiver and transmitter communicating with said computer such that said computer calculates the position of said drill guide relative to said surgical site.

21. The tracking system of claim 13, wherein said at least one guide sleeve comprises a plurality of guide sleeves having varying lengths.

22. The tracking system of claim 13, wherein said at least one guide sleeve includes an attachment, said attachment having a fixed and known position relative to said localizer assembly, such that said computer may calculate the position of said attachment and display a corresponding representation of said attachment relative to said surgical site.

23. A method for tracking a surgical procedure, comprising:
taking an image of a surgical site and storing said image on a computer system;
providing a surgical instrument;
providing an instrument guide having a localizer assembly and a handle assembly connected at a central bore and a guide sleeve having a tip end, an attachment end, and a sleeve bore, said guide sleeve having a fixed and known position relative to said localizer assembly;
front loading said attachment end of said guide sleeve into said central bore such that said tip end does not pass through said central bore;
calculating the position of said guide sleeve and displaying said position of said guide sleeve on said image relative to said surgical site by communication between said localizer assembly and said computer system; and
inserting said surgical instrument into said guide sleeve at said attachment end such that said surgical instrument emerges from said tip end to engage said surgical site.

24. The method of claim 23 wherein said guide sleeve includes a bore foot that is oriented to said guide sleeve at a desired angle such that said bore foot engages said surgical site in order that said guide sleeve is oriented to said surgical site at said desired angle.
